(19) United States
(12) Patent Application Publication
(10) Pub. No.: US 2007/0299026 A1
(43) Pub. Date: Dec. 27, 2007

(54) Immunosuppressive Cytokine

(76) Inventors: Foo Yew Liew, Glasgow (GB); Xiao-Qing Wei, Cardiff (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/593,247

(22) PCT Filed: Mar. 18, 2005
(86) PCT No.: PCT/GB05/01037

§ 371 Date: Jul. 2, 2007

(30) Foreign Application Priority Data
Mar. 18, 2004 (GB) 0406135.4
Apr. 5, 2004 (GB) 0407760.8

(51) Int. Cl. A61K 31/70; A61P 3/00; C07H 21/04; C12N 15/00

(52) U.S. Cl. 514/44; 435/320.1; 536/23.4

(57) ABSTRACT

The EB1-3-p35 cytokine is shown for the first time to be capable of inhibiting immune responses mediated or controlled by T cells via an effect on suppressor T cells. This suggests that EBI3-p35 may be therapeutically effective in a variety of inflammatory and autoimmune conditions including arthritis, atherosclerosis, allograft rejection, and allergies such as asthma.

### FIELD OF THE INVENTION

The present invention relates to cytokines, and in particular to the EBI3-p35 cytokine and its role in suppression of immune responses mediated or controlled by T cells.

### BACKGROUND TO THE INVENTION

IL-12 is a heterodimeric cytokine composed of two polypeptide subunits, p35 and p40, linked by disulphide bonds. IL-12 induces interferon-gamma production from NK cells, T cells, dendritic cells and macrophages as well as promoting differentiation of naive CD4+ T cells into type 1 helper T cells (Th1 cells) which also produce interferon-gamma (for review see Watford et al., Cytokine and Growth Factor Reviews 14 (2003) 361-368).

The two constituent polypeptide chains of IL-12 have different expression patterns, and each one has been shown to form heterodimers with polypeptides other than its partner in IL-12. For example, p40 has been shown to associate with a polypeptide known as p19 to form a cytokine designated IL-23, and in vitro can form homodimers which act as antagonists for IL-12 itself.

The p35 subunit, on the other hand, has been shown to associate with Epstein-Barr virus-induced protein 3 (EBI3), which has homology to IL-12 p40. EBI3 is also known to associate with another p35-like protein (designated p28) to form the cytokine IL-27. IL-27 is expressed in myeloid cells, in particular lipopolysaccharide-activated monocytes and monocyte-derived dendritic cells, and promotes proliferation of naive T cells. It has been suggested to polarize the immune response towards the Th1 type.

Thus a family of IL-12-like cytokines exists, having pleiotropic effects on cells of the immune system. To date, however, no function has been ascribed to the EBI3-p35 heterodimer.

The demonstration that EBI3-p35 is expressed in the placental syncytiotrophoblast, combined with the similarity of the heterodimer to IL-12, has led to the suggestion that the heterodimer may be in some way immunosuppressive (Devergne et al., Proc. Natl. Acad. Sci. USA 94 (1997) 12041-12046; WO97/13859). These authors proposed that any such activity would most likely be due to an antagonistic effect on IL-12 signalling, but provided no functional data in support of this speculation. As yet, then, the function of the EBI3-p35 molecule remains unknown.

### SUMMARY OF THE INVENTION

The present inventors have found that EBI3-p35 is capable of promoting proliferation of regulatory T cells (T_{R} or T_{reg} cells) in vitro. Regulatory T cells are known to play a significant role in the suppression of autoreactive T cells in vivo, and also to be capable of suppressing allograft rejection. Consistent with this known function of T_{reg} cells, the present inventors have further shown that EBI3-p35 has a substantial therapeutic effect in a mouse model of rheumatoid arthritis. Thus the present invention provides the first evidence of any physiological function for the EBI3-p35 cytokine.

Accordingly, in a first aspect, the present invention provides a method of stimulating proliferation of a regulatory T cell, comprising contacting the cell with EBI3-p35.

The EBI3-p35 may comprise at least two EBI3 components and/or at least two p35 components. A particularly preferred embodiment is a heterotetramer having two of each component.

In preferred embodiments, at least one EBI3 component and at least one p35 component are covalently linked to one another. Preferably the at least one EBI3 component and the at least one p35 component form a fusion protein.

Preferably each EBI3 or p35 component is covalently linked to at least one other EBI3 or p35 component. The EBI3-p35 may comprise one, two, or more fusion proteins, each comprising at least two of the EBI3 and p35 components. The fusion proteins may themselves be covalently linked by any appropriate means including a non-peptide chemical linker, a disulphide bond, etc. In preferred embodiments, all EBI3 and p35 components are covalently linked to one another.

The EBI3-p35 may further comprise one or more heterologous components, preferably heterologous polypeptides, covalently linked to one or more of the EBI3 or p35 components. The heterologous polypeptides may be part of a fusion protein with one or more EBI3 or p35 component.

The heterologous components are preferably capable of associating with one another and may hence assist in the association between the various EBI3 and p35 components. In such cases, the EBI3-p35 comprises two or more such heterologous components; the heterologous components may be the same or different.

The heterologous components may, additionally or alternatively, provide further biological effector functions. Particularly preferred heterologous components are polypeptides comprising antibody Fc sequences, which may be used to extend the half life of EBI3-p35 in vivo. Preferably antibody hinge sequences are also included, as these contain cysteine residues capable of forming disulphide bridges between Fc chains.

The method typically comprises contacting the regulatory T cell with a substance capable of stimulating signalling through the cell's T cell receptor complex. Examples of such substances include anti-CD3 antibodies and cells displaying antigens recognised by the T cell receptor in the context of a MHC molecule, including professional antigen presenting cells (APCs) such as dendritic cells, macrophages, etc. The APCs may themselves be prevented from proliferation, for example by fixation (e.g. with formaldehyde), irradiation (e.g. with X or gamma rays) or chemical treatment (e.g. with mitomycin C). Additionally or alternatively, co-stimulatory signals may be employed, such as anti-CD28 antibodies.

When the methods arc carried out in vitro or ex vivo (e.g. in cell culture), a TCR stimulus is normally provided to the T_{reg} cells along with the EBI3-p35. However the methods of the invention can also be performed in vivo, by administration of EBI3-p35 to a subject as a method of boosting the number of regulatory T cells in that subject. In such circumstances the T_{reg} cells within the body will normally receive sufficient TCR stimulus from their environment in vivo, and so will proliferate solely on administration of EBI3-p35.

When performed in vitro or ex vivo, the method may further comprise the step of formulating a population of regulatory T cells so obtained for administration to a subject. Preferably the recipient is syngeneic or histocompatible with the T cells. The recipient may have been the original source of the cells.

Thus the invention provides a method of providing a suppressor T cell obtained from a subject, contacting the cell in vitro with EBI3-p35 to produce a population of regulatory T cells, and formulating the population of regulatory T cells for administration to the subject. The method may additionally comprise the steps of obtaining the cells from and/or administering the cells to the subject.

In a further aspect, the invention provides the use of EBI3-p35, nucleic acid(s) encoding EBI3-p35, or cells expressing and secreting EBI-p35, in the manufacture of a medicament for increasing regulatory T cell activity in a subject. By this is meant increasing the number of regulatory T cells present in the subject and so increasing that subject's capacity to control effector T cell activity.

The invention further provides EBI3-p35, nucleic acid(s) encoding EBI3-p35, or cells expressing and secreting EBI-p35 for use in a method of medical treatment.

The invention further provides a method of enhancing regulatory T cell activity in a subject, comprising administering EBI3-p35, nucleic acid(s) encoding EBI3-p35, or cells expressing and secreting EBI3-p35, to that subject.

Such preparations and methods may be used in the treatment of conditions characterised by inappropriate or undesirable T cell activation, including inflammatory or autoimmune diseases. They may also be used for the prevention of allograft rejection or prolonging allograft survival.

Particular conditions which may be treated by the methods and compositions of the invention include allergy (e.g.) asthma, arthritis (e.g. rheumatoid arthritis), gastritis, pernicious anaemia, thyroiditis, insulitis, diabetes, sialoadenitis, adrenalitis, autoimmune orchitis/oophoritis, glomerulonephritis, experimental autoimmune encephalitis, multiple sclerosis, inflammatory bowel disease, atherosclerosis and chronic obstructive pulmonary disease.

Preferred characteristics of the EBI3-p35 for use in such compositions and methods are set out above in relation to the first aspect of the invention, and elsewhere in this specification.

In a further aspect the present invention provides an EBI3-p35 molecule comprising an EBI3 component, a p35 component, and a heterologous component, wherein two or more of the heterologous components are capable of associating with one another such that two or more such EBI3-p35 molecules form a complex.

Preferably the EBI3-p35 molecule is a fusion protein comprising EBI3, p35 and heterologous components. In preferred embodiments the heterologous components are capable of associating with one another by formation of disulphide bonds. A particularly preferred example of such a heterologous component is an antibody Fc sequence including hinge sequence.

The present invention further provides EBI3-p35 comprising two EBI3 components and two p35 components. Preferably each of the components is covalently linked to at least one other component of the complex. The complex may comprise one or more fusion proteins, each comprising at least two said components, preferably at least one EBI3 component and at least one p35 component. Such fusion proteins may further comprise one or more heterologous components as described above.

The invention further provides a nucleic acid encoding a fusion protein as described in any of the aspects of the invention above. Also provided is an expression vector comprising a nucleic acid of the invention and a host cell comprising an expression vector of the invention.

### BRIEF DESCRIPTION OF THE DRAWINGS

FIG. 1. (A) Western blot analysis of EBI3-p35-Fc using anti-human Fc antibody. A clear band was detected at MW 78 kDa. (B) Schematic representation of the fusion protein EBI3-p35-Fc. The protein is likely to form a homodimer through the disulphide bonds at the Fc/hinge region.

FIG. 2 shows Coomassie blue staining of purified EBI-p35-Fc. A single band at 78 kDa is shown in all lanes. (2 fold dilution of protein starting at 1 μg/lane)

FIG. 3. Effect of EBI3-p35-Fc on the proliferation of CD4+ and CD4+CD25+ T cells in vitro. Cells were purified from BALB/c mice and cultured with plate-bound anti-CD3 antibody and graded concentrations of the fusion protein. Cellular proliferation was determined by ³H-Thymidine incorporation at 72 h and expressed as counts per minute.

FIG. 4 shows the effect of EBI3-p35-Fc on the proliferation of CD4⁺CD25⁺ T_{reg} cells. CD4⁺CD25⁺ T cells were purified from BALB/c mice and cultured with plate-bound anti-CD28 and anti-CD3 in the presence of IL-2. EBI3-p35-Fc was added at varying concentrations and proliferation was measured by ³H-thymidine incorporation.

FIG. 5 shows that EBI3-p35-Fc expanded CD4+CD25+ T_{reg} cells retain suppressive function against CD4+CD25− T cells. Cells were purified from BALB/c lymph nodes and cultured with plated-bound anti-CD3 antibody for 3 days. The cells were washed and then cultured either alone or in 1:1 ratio for a further 3 days in the presence of soluble anti-CD3 antibody and Mytomycin C-treated antigen presenting cells. Cellular proliferation was determined by 3H-thymidine incorporation and expressed as cpm, n=6. FIG. 6 shows therapeutic effect of EBI3-p35-Fc in collagen-induced arthritis in mice. Groups of 10 male DBA/1 mice (6-8 weeks old) were immunised subcutaneously with 200 μg of bovine type II collagen (CII) in Freund's complete adjuvant and boosted intraperitoneally (i.p.) 21 days later with 200 μg of CII in PBS. The mice were treated i.p. daily with 2 μg of EBI3-p35-Fc or PBS from day 24-day 30. Mice were monitored daily for disease symptoms. Vertical bars represent Mean±SEM.

FIG. 7 shows the complete amino acid sequence (including signal peptide) of the EBI3-p35-Fc fusion protein described in the Examples.

FIG. 8 shows that EBI3-p35-Fc is capable of attenuating established arthritis. DBA/1 mice were primed and boosted with type II collagen in FCA as described in relation to FIG. 6, above. The mice were then treated intraperitoneally daily with 2 μg EBI3-p35-Fc, Enbrel, EBI3-p35-Fc plus Enbrel or sIL-15R from days 27-36. Control mice were given PBS only.

FIG. 9 shows the effect of EBI3-p35-Fc on a murine model of asthma. BALB/c mice were injected intraperitoneally with 100 μg of chicken ovalbumin (OVA) in an alum suspension on days 0 and 14. On day 14, mice were anaesthetised with avertin, and 100 μg of OVA in 40 μl of PBS was administered intratracheally (i.t.). Mice were again anaesthetised before being challenged i.t. on each of the days 25, 26, and 27 with 10 μg OVA in 40 μl of PBS. For EBI3-p35 treatment, mice were injected intraperitoneally with 2 μg of EBI3-p35 on three occasions (day 25, 26 and 27) 1 h before the OVA challenge. Negative control mice were given PBS in place of OVA in both the sensitisation and the challenge stages. Mice were sacrificed on day 29 by administration of a fatal dose of avertin. Immediately after the administration of avertin, blood sample was collected by cardiac puncture. Bronchoalveolar lavage (BAL) was then harvested and counted using a haemocytometer. Total cell counts are shown in panel (A). Specific ELISA assays were used to determine the concentration of ovalbumin-specific IgE (Panel (B)) and IL-4 (Panel (C)) in the BAL supernatant.

### DETAILED DESCRIPTION OF THE INVENTION

EBI3 and p35

The human EBI3 (Epstein-Barr virus-induced gene 3) gene encodes a protein of approximately 33 kDa with approximately 27% amino acid sequence identity to the p40 subunit of human IL-12. Exemplary nucleic acid and amino acid sequences for human EBI3 are provided as SEQ ID NOs 1 and 2 respectively of WO97/13859. Exemplary sequences for murine and human EBI3 are also provided in GenBank as accession numbers NM015766 and BC046112 respectively.

References to EBI3 components of EBI3-p35 should be taken to include polypeptides having those sequences, or sequences encoded by those nucleic acid sequences (with or without signal peptide), as well as wild-type EBI3 polypeptides encoded by orthologous genes from other species, and polypetides having sufficient sequence identity to those polypeptides to retain the ability to stimulate proliferation of regulator T cells from the same species when provided in a heterotetramer with a suitable p35 component.

Thus preferred EBI3 polypeptide sequences have at least 80% sequence identity, preferably at least 85% sequence identity, more preferably at least 90%, 91%, 92%, 93%, 94%, 95%, 96%, 97%, 98% or 99% sequence identity to the wild-type sequences referred to above (e.g. those from WO97/13859 or GenBank, or to wild-type polypeptides encoded by orthologous genes from other species). Reference to a nucleic acid encoding an EBI3 polypeptide should be construed accordingly.

p35 was originally identified as a component of the cytokine IL-12. Exemplary nucleic acid and amino acid sequences for human p35 are provided as SEQ ID NOs 3 and 4 respectively of WO97/13859. Exemplary sequences for human and murine p35 are also found at GenBank accession numbers NM_{—}000882 and M86672 respectively. References to p35 components of EBI3-p35 should be taken to include polypeptides having those sequences, or sequences encoded by those nucleic acid sequences (with or without signal peptide), as well as wild-type p35 polypeptides encoded by orthologous genes from other species, and polypetides having sufficient sequence identity to those polypeptides to retain the ability to stimulate proliferation of regulator T cells from the same species when provided in a heterotetramer with a suitable EBI3 component.

Thus preferred p35 polypeptide sequences have at least 80% sequence identity, preferably at least 85% sequence identity, more preferably at least 90%, 91%, 92%, 93%, 94%, 95%, 96%, 97%, 98% or 99% sequence identity to the sequences referred to above (e.g. those from WO97/13859 or GenBank, or to wild-type polypeptides encoded by orthologous genes from other species). Reference to a nucleic acid encoding a p35 polypeptide should be construed accordingly.

In particular, conservative substitutions in EBI3 or p35 (as compared to the reference sequences) may be particularly well tolerated, without substantial effect on EBI3-p35 function.

A conservative substitution may be defined as a substitution within an amino acid class and/or a substitution that scores positive in the BLOSUM62 matrix.

According to one classification, the amino acid classes are acidic, basic, uncharged polar and nonpolar, wherein acidic amino acids are Asp and Glu; basic amino acids are Arg, Lys and His; uncharged polar amino acids are Asn, Gln, Ser, Thr and Tyr; and non-polar amino acids are Ala, Gly, Val, Leu, Ile, Pro, Phe, Met, Trp and Cys.

According to another classification, the amino acid classes are small hydrophilic, acid/acidamide/hydrophilic, basic, small hydrophobic and aromatic, wherein small hydrophilic amino acids are Ser, Thr, Pro, Ala and Gly; acid/acidamide/hydrophilic amino acids are Asn, Asp, Glu and Gin; basic amino acids are His, Arg and Lys; small hydrophobic amino acids are Met, Ile, Leu and Val; and aromatic amino acids are Phe, Tyr and Trp

Substitutions which score positive in the BLOSUM62 matrix are as follows:

| | Original residue | | | | | | | | | | | | | | | | | | | |
|---|---|---|---|---|---|---|---|---|---|---|---|---|---|---|---|---|---|---|---|---|
| | C | S | T | P | A | G | N | D | E | Q | H | R | K | M | I | L | V | F | Y | W |
| Substi- | — | TAN | S | — | S | — | SDH | NE | DQK | ERK | NY | QK | EQR | ILV | MLV | MIV | MIL | YW | HFW | FY |
| tution | | | | | | | | | | | | | | | | | | | | |

Percent (%) amino acid sequence identity with respect to a reference sequence is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. % identity values may be determined by WU-BLAST-2 (Altschul et al., Methods in Enzymology, 266:460-480 (1996)). WU-BLAST-2 uses several search parameters, most of which are set to the default values. The adjustable parameters are set with the following values: overlap span=1, overlap fraction=0.125, word threshold (T)=11. A % amino acid sequence identity value is determined by the number of matching identical residues as determined by WU-BLAST-2, divided by the total number of residues of the reference sequence (gaps introduced by WU-BLAST-2 into the reference sequence to maximize the alignment score being ignored), multiplied by 100.

Percent (%) amino acid similarity is defined in the same way as identity, with the exception that residues scoring a positive value in the BLOSUM62 matrix are counted. Thus, residues which are non-identical but which have similar properties (e.g. as a result of conservative substitutions) are also counted.

In a similar manner, percent (%) nucleic acid sequence identity with respect to a reference nucleic acid is defined as the percentage of nucleotide residues in a candidate sequence that are identical with the nucleotide residues in the reference nucleic acid sequence. The identity values used herein may be generated by the BLASTN module of WU-BLAST-2 set to the default parameters, with overlap span and overlap fraction set to 1 and 0.125, respectively.

All accession numbers provided in this specification are taken from GenBank release no. 140.0; 15 February 2004.

EBI3-p35

The term EBI3-p35 as used herein refers to any intramolecular complex or single molecule comprising at least one EBI3 polypeptide component and at least one p35 polypeptide component as described above. EBI3 and p35 are known to associate with one another in vivo; according to Devergne et al. (see above) this interaction is non-covalent, not involving disulphide bonds.

As used in the present invention, the EBI3 and p35 components may be associated with one another either covalently or non-covalently. Covalent association may be desirable, as the EBI3-p35 molecule thus formed may have benefits over a non-covalently associated complex in terms of stability and possibly also activity.

In preferred embodiments, the EBI3 and p35 components are coexpressed as a fusion protein. To produce a fusion protein, a nucleic acid expression vector is constructed comprising coding sequences for each component in one continuous open reading frame, so that the two components can be translated as part of the same polypeptide chain.

Typically, a flexible peptide linker is included between the two components to allow the two components to interact freely with one another without steric hindrance. The skilled person is perfectly capable of designing a suitable linker. Conventionally, such linkers are between 12 and 20 amino acids in length, and have a high proportion of small and hydrophilic amino acid residues (e.g. glycine and serine) to provide the required flexibility without compromising aqueous solubility of the molecule.

Alternatively, one or both of the EBI3 and p35 components may be engineered to increase their affinity for one another. This may be achieved in various ways. For example, cysteine residues may be introduced into one or both components to enable the two components to form disulphide bonds with one another.

As a further alternative, interaction between the EBI3 and p35 components may be promoted by linking each component to a heterologous component, wherein the two heterologous components are capable of interacting with one another. Where the heterologous components are polypeptides, they may be expressed as fusion proteins with the EBI3 and p35 components.

Preferred heterologous components are polypeptides comprising antibody Fc sequences, and preferably one or more antibody Fc domains (e.g. CH2, CH3 and/or CH4 domains (if appropriate) of IgG, IgM, etc.). Preferably the hinge sequence normally located between the CH1 and CH2 domains is also included. The hinge region contains cysteine residues which form disulphide bonds between the heavy chains of the intact native antibody. Thus if the hinge regions are present in the EBI3-p35 molecules described herein, similar bonds will be formed to stabilise the interactions between the chains.

The skilled person will be aware of alternative heterologous components which may be used to increase or stabilise the interaction between EBI3 and p35 components. These include leucine zipper polypetides, which dimerise via hydrophobic interactions.

Although recombinant methods are preferred, EBI3 and p35 components may also be covalently linked by chemical means. Bifunctional and polyfunctional chemical linker molecules suitable for conjugating or cross-linking polypeptide molecules to one another are well known to the skilled person.

EBI3-p35 complexes and molecules as used in the methods and compositions of the invention may comprise two or more EBI3 components and/or two or more p35 components. In particularly preferred embodiments, EBI3-p35 comprises two EBI3 components and two p35 components; i.e. it is a heterotetramer.

Preferably each EBI3 and p35 component is covalently linked to at least one other EBI3 or p35 component. More preferably, all EBI3 and p35 components in the EBI3-p35 molecule are covalently linked to one another. Such covalent links may be direct (i.e. between atoms of EBI3 and p35 components) or indirect (e.g. via chemical linkers, via peptide linkers in fusion proteins, or via disulphide links between heterologous components which are themselves covalently linked to one or more of the EBI3 or p35 components).

The skilled person will be able to conceive of numerous possible configurations for the EBI-p35 molecule. By way of example, the EBI3-p35 molecule may be a single polypeptide chain comprising (at least) two EBI3 and two p35 components. Alternatively it may comprise (at least) two fusion proteins, each comprising a p35 and an EBI3 component, which may be covalently or non-covalently joined together, e.g. via heterologous components of the fusion proteins. The construct described in the examples comprises two polypeptides, each made up of a p35 component, an EBI3 component and an antibody hinge/Fc sequence. The two chains are covalently joined via disulphide bonds formed between the antibody hinge regions.

Heterologous components may also be used to impart additional or improved properties to the EBI3-p35. For example, fusion proteins comprising antibody Fc and hinge regions (commonly referred to as immunoadhesins) typically have a longer half life in vivo than the proteins alone. Thus the construct described in the Examples may have an improved half life in vivo as compared to a EBI3-p35 heterotetramer lacking the Fc region. Such an immunoadhesin-type construct may require less frequent administration to a patient than other proteins. Preferably the Fc and hinge regions are derived from an IgG molecule.

Regulatory T Cells

Regulatory T cells (T_{R} or T_{reg} cells; Sakaguchi S et al. J. Immunol. 155:1151, 1995) are a subset of T cells whose major function appears to be to downregulate the proliferation and activity of autoreactive effector T cells. For reviews see Shevach EM Ann. Rev. Immunol. 18:423, 2000; Maloy K and F Powrie. Nat. Immunol. 2:816, 2001; Sakaguchi S et al. Immunol. Rev.182:18, 2001.

T_{R} cells are typically CD4+CD25+, although the transcription factor FOXP3 (Brunkow, M. E. et al. (2001). Nat. Genet. 27:68-73) may be a more reliable marker for committed T_{R} cells than CD25 (Hori et al. (2003) Science. 299:1057-1061; Walker et al. (2003) J. Clin. Invest. 112:1437-1443). Thus the term “regulator T cell” may be taken to mean a T cell expressing at least CD4 and FOXP3, and optionally also CD25.

Humans with mutations in FOXP3 and so deficient in T_{R} cells suffer from IPEX (immune dysregulation, polyendocrinopathy, enteropathy, X-linked syndrome) which is accompanied by autoimmune disease such as type I diabetes, inflammatory bowel disorder and severe allergy. Although it is not envisaged that simple administration of EBI3-p35 would treat IPEX itself, it is clear that the associated conditions may be caused by inadequate activity or dysfunction of T_{reg} cells. Therefore EBI3-p35 should be useful for treatment of the similar conditions (e.g. type I diabetes, inflammatory bowel disorder and allergy, such as asthma) in subjects which are capable of producing functional T_{R} cells.

Depletion of T_{R} cells or impairment of T_{R} cell function has been shown to result in autoimmune disease in murine models. Disease caused in test animals include arthritis (e.g. rheumatoid arthritis), inflammatory bowel disease, gastritis, pernicious anaemia, thyroiditis, insulitis, diabetes, sialoadenitis, adrenalitis, autoimmune orchitis/oophoritis, glomerulonephritis, chronic obstructive pulmonary disease and experimental autoimmune encephalitis and multiple sclerosis.

Induction of a regulatory T cell type 1 response has also been shown to reduce the development of atherosclerosis in murine models (Mallat Z. et al. Circulation 108:1232-7, 2003).

T_{R} activity has also been shown to be significant in the rate at which allografts are rejected. Depletion of T_{R} cells or impairment of function accelerates the rate of rejection, while infusion of test animals with syngeneic lymphocytes enriched in T_{R} cells has been shown to prolong graft survival. Thus EBI3-p35 may also be used to treat graft rejection or prolong graft survival.

Therapy using EBI3-p35

In view of the above, it will be seen that EBI3-p35 therefore represents a realistic therapeutic for treatment of the above mentioned conditions and for prolonging graft survival, e.g. in transplant recipients, by boosting the number of regulatory T cells in affected subjects and so increasing their capacity to downregulate activity of effector T cells (e.g. helper and cytotoxic T cell).

EBI3-p35 protein may be administered directly to subjects in pharmaceutical compositions.

Alternatively, nucleic acids encoding EBI3-p35 constructs may be administered to subjects such that EBI3-p35 is expressed from the subject's own cells. Typically the nucleic acids will be part of one or more expression vectors, which may be administered as naked nucleic acid or in a delivery vehicle such as viral vector.

As an alternative, cells which are naturally capable of expressing and secreting EBI3-p35, or which have been engineered to do so, may be administered to a subject. Preferably the cells are syngeneic or histocompatible with the subject. For example, cells may be removed from a subject, transfected with one or more suitable vectors, and readministered to the subject.

The skilled person will be capable of designing suitable nucleic acid expression vectors for therapeutic uses (as well as for other uses described in this specification). The vectors will typically contain appropriate regulatory sequences, including promoter sequences, terminator fragments, enhancer sequences, marker genes and other sequences, depending upon the particular form of EBI3-p35 which is to be administered (see above). The vectors may be intended to integrate into a host cell chromosome, or may exist and replicate independently of the host chromosomes as an episome.

Where the EBI3-p35 to be expressed is composed of two discrete (i.e. independently transcribed and translated) polypeptide chains, these polypeptides will normally be encoded by discrete genes or expression cassettes. These genes or expression cassettes may be located on the same vector, i.e. as part of a single nucleic acid molecule, or on separate vectors.

EBI3-p35 can be used in vivo to increase the number of regulatory T cells in a subject. However EBI3-p35 can also be used in vitro or ex vivo (e.g. in cell culture) to expand populations of regulatory T cells.

Regulatory T cells may be isolated from a sample (e.g. of blood or peripheral blood mononuclear cells) prior to treatment with EBI3-p35 (e.g. by selection for CD4+CD25+ lymphocytes, by magnetic cell sorting or other suitable methods), or alternatively a heterogeneous population of lymphocytes may be treated with EBI3-p35. The expanded population of regulatory T cells may thereafter be purified further if desired.

Thus populations of lymphocytes may be enriched in regulatory T cells, or more or less pure populations of regulatory cells may be generated in the laboratory. The cells so obtained may be useful for research purposes or for administration to a subject.

Regulatory T cells obtained in the laboratory by such methods can therefore be formulated appropriately (e.g. in a pharmaceutical composition) for administration to a subject, who is preferably syngeneic or histocompatible with those cells.

Preferably, the recipient will have been the original source of the cells. A sample of blood containing regulatory T cells can therefore be obtained from a subject, before expanding the regulatory T cells to a desired degree by treatment with EBI3-p35 and readministering the expanded cells to the subject. This may be useful if for any reason it is not feasible to administer EBI3-p35 protein directly to the subject.

Preferred subjects for treatment by the methods of the invention are mammals. Preferred subjects are primates (including humans), rodents (including mice and rats), and other common laboratory, domestic and agricultural animals, including but not limited to rabbits, dogs, cats, horses, cows, pigs, sheep, goats, etc.

Pharmaceutical Formulations

The complexes, polypeptides, nucleic acids and cells of the invention can be formulated in pharmaceutical compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular and intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection.

Preservatives, stabilisers, buffers, antioxidants and/or other additives may be included, as required.

Whatever the nature of the active agent that is to be given to an individual (e.g. a cell, polypeptide, nucleic acid molecule, other pharmaceutically useful agent according to the present invention), administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions or dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they could be produced in the target cells by expression from an encoding gene introduced into the cells, e.g. in a viral vector (e.g. a retroviral, lentiviral or adenoviral vector). The vector could be targeted to the specific cells to be treated, or it could contain regulatory elements which are switched on more or less selectively by the target cells.

A composition may be administered alone or in combination with other treatments, either simultaneously or sequentially dependent upon the condition to be treated.

### EXAMPLES

### 1. Construction of pSec-Linker Vector

Two complementary oligos were designed and synthesised with 5′ end phosphorylation. Sense: 5′-GATCC GGT GGT GGT GGT TCT GGT GGT GGT GGT TCT GGT GGT GGT GGT TCT G; anti-sense: 5′-AATTC AGA ACC ACC ACC ACC AGA ACC ACC ACC ACC AGA ACC ACC ACC ACC G. The restriction enzyme sites BamHI and EcoRI were introduced at 5′ and 3′ ends respectively. Oligos were dissolved with TE buffer and adjusted to 120 μmol/μl. 100 μl of each were mixed and incubated in a heat block at 98° C. for 10 minutes, followed by cooling down naturally to room temperature to anneal the two oligos. This dsDNA fragment encodes a linker sequence in a reading frame shown as below.

```
5′-GATCC GGT GGT GGT GGT TCT GGT GGT GGT GGT TCT GGT GGT GGT GGT TCT G
              G CCA CCA CCA CCA AGA CCA CCA CCA CCA AGA CCA CCA CCA CCA AGA CTTAA-5′
      G S G G G G S G G G G S G G G G S E K
```

A vector pSecTag2A (Invitrogen) was digested with BamHI and EcoRI before purified from agarose gel with a gel extraction kit (QIAGEN). Linker fragments were ligated with digested vector pSecTag2A before transformation of DH5(competent cells with 5 μl of ligation reaction. Two plasmids from individual clones were DNA sequenced. Both of them contained a single copy of linker sequence in the correct orientation.

### 2. Construction of pSec-Linker-hIgG1Fc Vector

PCR-amplified hIgG-Fc/hinge fragment from human PBMC was reverse-transcribed to cDNA. The primers used were: Sense: 5′-GAG CCT CGA GCC GAG CCC AAA TCT TGT GA; antisense: 5′-AGA AGT CGA CTT ATT TAC CCG GGG ACA GG. Purified PCR product was digested with XhoI and Sali and further purified in agarose gel. At the same time, the pSec-Linker vector was prepared by digestion with XhoI and dephosphorylated with Shrimp AP before purified from gel as described above. Ligation was set up for overnight at 15° C. with human IgG Fc PCR fragment and pSec-Liker vector. The pSec-Linker-hIgG1Fc vector was purified from transformed DH5α.

### 3. Construction of EBI3-p35-Fc Expression Plasmid

The fragments for EBI3 and IL-12p35 open reading frames were amplified by RT-PCR, respectively, from total RNA of murine bone marrow macrophage after stimulation overnight with LPS and IFNγ. The PCR fragments were inserted into TA vector (Invitrogen) for DNA sequencing. Sequencing result matched exactly with Genebank sequences for murine EBI3 and IL-12p35.

The following PCR primers were designed to construct the expression vector of EBI3-IL-12p35-hFc:

EBI3 sense:
5′-CCCCGGATCCCACTGAAACAGCTCTCGTGGCTCT
EBI3 antisense:
5′-CGGGATCCCTTATGGGGTGCACTTTCTACTTGCC
IL-12p35 sense:
5′-GGCCGAATTCATTCCAGTCTCTGGACCTGCCA
IL-12p35 antisense:
5′-GGCGGCGGCCGCATAGCCCATCACCCTGTTGA

PCR fragments coding for EBI3 and IL-12p35 proteins were amplified with primers above and pfu DNA polymerase from the EBI3 and p35 cDNA TA vector clones. EBI3 PCR fragment was digested with BamHI and inserted into pSec-Linker-hIgGlFc vector BamHI site as shown below.

```
ATG GAG ACA GAC ACA CTC CTG CTA TGG GTA CTG CTG CTC TGG GTT
Met Glu Thr Asp Thr Leu Leu Leu Trp Val Leu Leu Leu Trp Val
                                                        Ig-κ-chain leader sequence
CCA GGT TCC ACT GGT GAC GCG GCC CAG CCG GCC AGG CGC GCC GTA
Pro Gly Ser Thr Gly Asp Ala Ala Gln Pro Ala Arg Arg Ala Val
                                                            BamHI
CGA AGC TTG GTA CCG AGC TCG| GAT CC
Arg Ser Leu . . .
```

The orientation of insertion was checked by EcoRV digestion. This vector was designated pEBI3-L-Fc. pEBI3-L-Fc was opened with EcoRI and NotI before gel purification. mIL-12p35 PCR fragment was also digested with EcoRI and NotI and inserted into the pEBI3-L-Fc vector to produce pEBI3-L-p35-Fc (below)

```
BamHI
GGA TCC GGT GGT GGT GGT TCT GGT GGT GGT GGT TCT
Gly Ser Gly Gly Gly Gly Ser Gly Gly Gly Gly Ser
                                                                  Linker
                                          EcoRI
GGT GGT GGT GGT TCT GAA TTC . . . p35 . . .
Gly Gly Gly Gly Ser Glu Phe
EcoRI Pst I NotI
GA ATT CTG CAG ATA TCC AGC ACA GTG GCG GCC GCT
    Xho I
CGA GCC GAG CCC AAA TCT TGT . . . hIgG-hingeFc
Arg Ala Glu Pro Lys Ser Cys
```

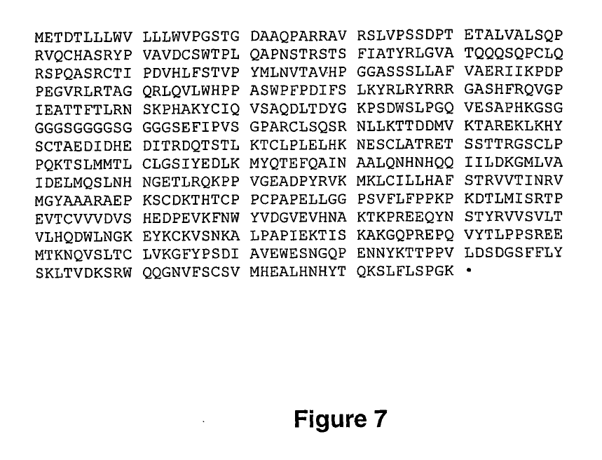

The full-length amino acid sequence of the EBI3-p35-Fc fusion protein is shown in FIG. 7.

### 4. Expression of EBI3-p35-Fc in Mammalian Cells

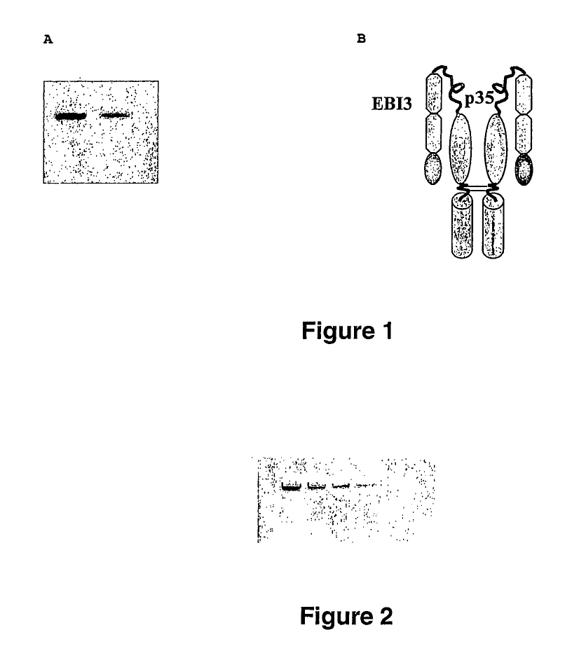

Cos-7 cells were transfected with the expression vector EBI3-p35-Fc and the protein produced was detected after 48 h by hIgG ELISA. The expressed protein was precipitated with protein A agarose beads before Western blotting with anti-human IgG1 antibody. A protein band at the predicted molecular weight (78 kDa) is shown in FIG. 1 which also shows a diagrammatic representation of the fusion protein.

CHO cells were transfected with vector EBI3-p25-Fc and Zeocin resistant cells were selected after two weeks. Twenty colonies were picked for expansion. Three colonies expressing the highest level of recombinant protein were retained for further use.

### 5. Purification of EBI3-p35-Fc by Affinity Column

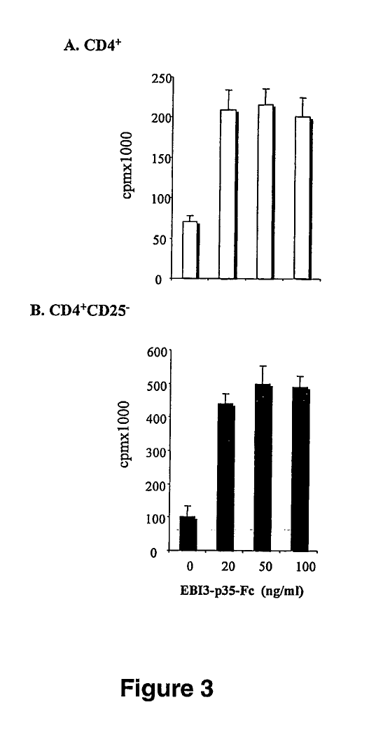

One of the higher expression cell lines was expanded with 10% ultra low IgG foetal bovine serum (GIBCO) DMEM medium. One litre of medium was harvested after 9 days. The culture supernatant was centrifuged at 5000 rpm for 30 minutes to get rid of cell debris and then loaded onto a protein agarose column at 4° C. overnight. The flow speed was kept below 1 ml per minute to allow the fusion protein to bind to the protein A beads. The beads in the column were washed at room temperature with PBS until the OD280 of the flow-through was below 0.01. The bound protein was eluted with elution buffer (0.1 M Glycine, pH 3.0). 15×1 ml fractions were collected and neutralised immediately with 50 μl of 2M Tris.HCl, pH 8.0. The protein concentration of each fraction was measured with a Coomassie Protein Assay. Those fractions containing higher concentrations of protein were pooled. The protein purity was checked with SDS-PAGE (FIG. 2).

### 6. Functional analysis of EBI3-p35-Fc In vitro

We first investigated the ability of EBI3-p35-Fc to induce T cell proliferation in vitro.

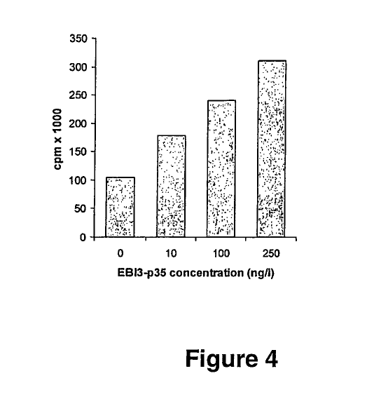

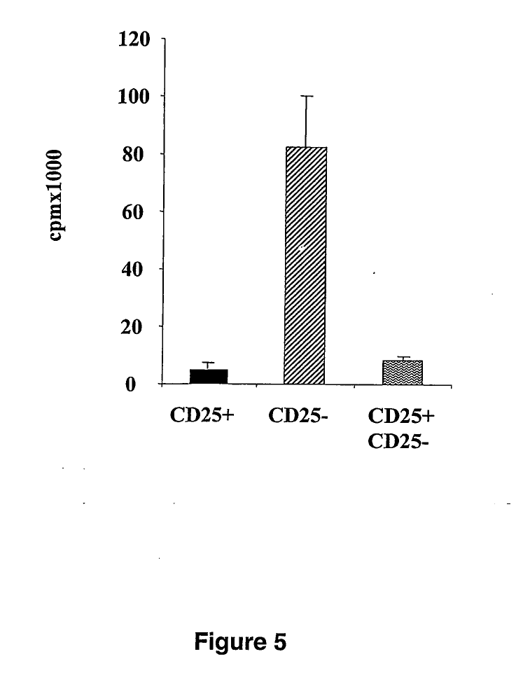

T cells were purified from the spleen and lymph nodes of normal BALB/c mice by magnetic adherence cell sorting (MACS). They were then further sorted into CD4⁺, CD8⁺, CD4⁺CD25⁻ and CD4⁺CD25⁺ subsets. The purity of the cells was normally>95% demonstrated by flow cytometry (not shown). Total CD4⁺ T cells and CD4⁺CD25⁻ T cells were cultured for 72 h with plate-bound anti-CD3 antibody (1 μg/ml) in culture medium in the presence of graded concentrations of EBI3-p35-Fc. Both subsets of T cells proliferated without the addition of EBI3-p35-Fc, which induced further proliferation of these cells in a dose-dependent manner (see for example FIG. 3). CD4⁺CD25⁺ T cells are known as regulatory T (T_{reg}) cells (Sakaguchi S et al. J. Immunol. 155:1151, 1995). Their major function is to down regulate the expansion of effector cells such as CD4⁺CD25⁻ T cells, and CD8⁺ T cells, the excessive activation of which can lead to a range of autoimmune disease (Shevach EM Ann. Rev. Immunol. 18:423, 2000; Maloy K and F. Powrie F. Nat. Immunol. 2:816, 2001). CD4⁺CD25⁺ T cells are naturally occurring and are notoriously difficult to expand in vitro. FIG. 4 shows that CD4⁺CD25⁺ T cells proliferated in the presence of EBI3-p35-Fc in a dose-dependant manner. These T_{reg} cells are powerful regulators which can suppress the proliferation of CD4⁺CD25⁻ T effector cell at a ratio of 1:10. We therefore wondered if a function of EBI3-p35-Fc in vivo is the expansion of T_{reg} cells, hence preventing the over expansion of effector T cells such as CD4⁺CD25⁻ T cells.

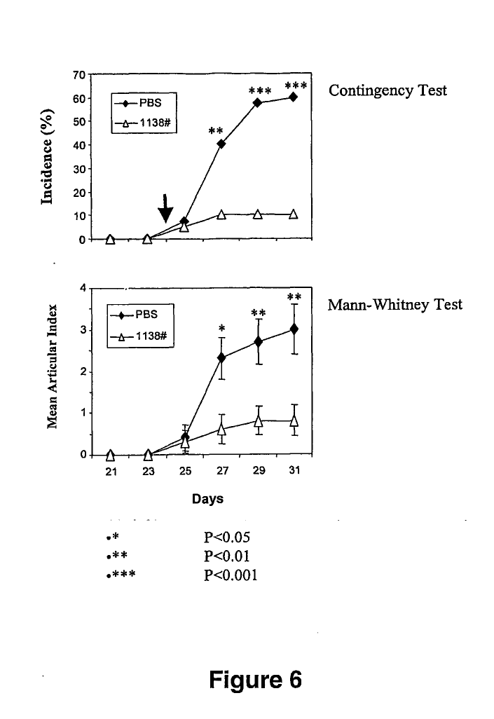

CD4⁺CD25⁺ and CD4⁺CD25⁻ T cells were purified from BALB/c mice as above and then cultured with plate-bound anti-CD3 antibody in the presence of EBI3-p35-Fc for three days. The cells were harvested, washed and cultured with soluble anti-CD3 and antigen-presenting cells to test the suppressive activity of the CD4⁺CD25⁺ T_{reg} cells. The two subsets of expanded T cells were cultured either alone or in combination (in a 1:1 ratio). FIG. 5 shows that while CD4⁺CD25⁻ T cells alone proliferated significantly under these conditions, CD4⁺CD25⁺ T cells did not. Interestingly, the expanded CD4⁺CD25⁺ T_{reg} cells suppressed the proliferation of CD4⁺CD25⁻ effector cells. Similar suppression of cytokine (IL-2 and IFNγ) production was also observed (data not shown). These data suggest that EBI3-p35-Fc may have therapeutic potential in treating autoimmune diseases by expanding CD4⁺CD25⁺ T_{reg} cells in vivo.

### 7. Effect of EBI3-p35-Fc in vivo

We have tested the effect of EBI3-p35-Fc in the collagen-induced arthritis (CIA) model in the mouse using an established protocol (Leung BP et al. J. Immunol. 170:1524, 2003). CIA is generally accepted as a surrogate for rheumatoid arthritis, a disease afflicting up to 1% of the human population world wide. In this model, DBA/1 mice were immunized subcutaneously with bovine type II collagen (CII) (200 μg) in Freund's complete adjuvant and boosted intraperitoneally on day 21 with CII (200 μg) in phosphate buffered saline (PBS). Mice were injected intraperitoneally daily with PBS or EBI3-p35-Fc (2 μg/mouse) from day 24 when symptom of arthritis began. Mice were monitored daily for signs of arthritis, for which severity scores were derived as follows: 0=normal, 1=erythema, 2=erythema plus swelling, 3=extension/loss of functions, and total score=sum of four limbs. Paw thickness was measured with a dial-caliper (Kroeplin, Munich, Germany). FIG. 6 shows that control mice treated with PBS developed the expected disease (incident and clinical score), whereas those treated with EBI3-p35-Fc showed minimum disease symptom. While the control mice became malaise and showed significant weight loss, the treated mice remained healthy and show normal weight gain. These results therefore clearly illustrate the therapeutic potential of EBI3-p35-Fc in inflammatory and/or autoimmune disease.

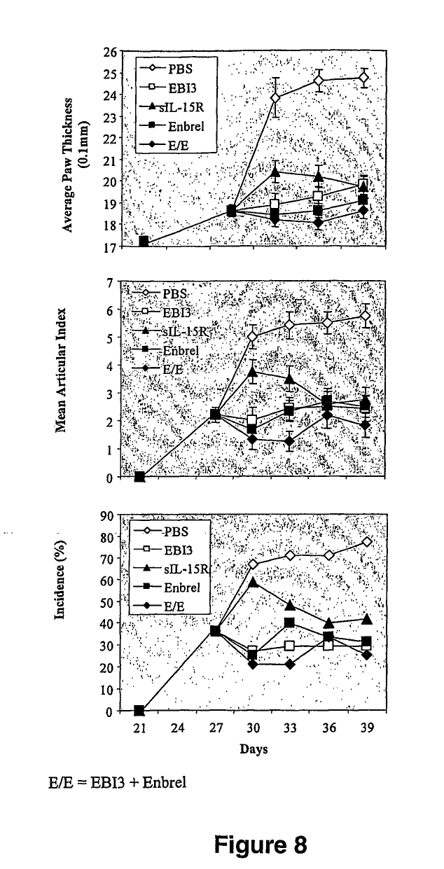

We next tested the ability of EBI3-p35-Fc to attenuate an established arthritis, and compared this effect with that of reagents known to be beneficial to clinical (recombinant TNF-R alpha (Enbrel)) and experimental (sIL-15Rα) arthritis. DBA/1 mice were primed and boosted with type II collagen in FCA as described above. The mice were then treated ip daily with 2 μg EBI3-p35-Fc, Enbrel, a combination of the two reagents, or sIL-15R, from days 27-36. As shown in FIG. 8, EBI3-p35-Fc is effective in attenuating ongoing CIA when injected intraperitoneally on day 27 of the experiment when the disease is already established. Furthermore, EBI3-p35-Fc is as effective as sIL-15Rα or Enbrel in the treatment of CIA.

### 8. Potential Therapeutic Role of EBI3-p35-Fc in Other Diseases

CD4⁺CD25⁺ T_{reg} cells have a broad range of suppressive activities. In experimental murine models, These T_{reg} cells have been shown to suppress CIA, asthma, gastritis, inflammatory bowel disease and allograft rejections (Sakaguchi S et al. Immunol. Rev.182:18, 2001; Shevach E M Ann. Rev. Immunol. 18:423, 2000). Based on our discovery that EBI3-p35-Fc powerfully expanded CD4⁺CD25⁺ T_{reg} cells in vitro and their demonstrated therapeutic effect in CIA, it is expected that this fusion molecule and other EBI3-p35 complexes and constructs as described in this specification would have a therapeutic role against all these conditions.

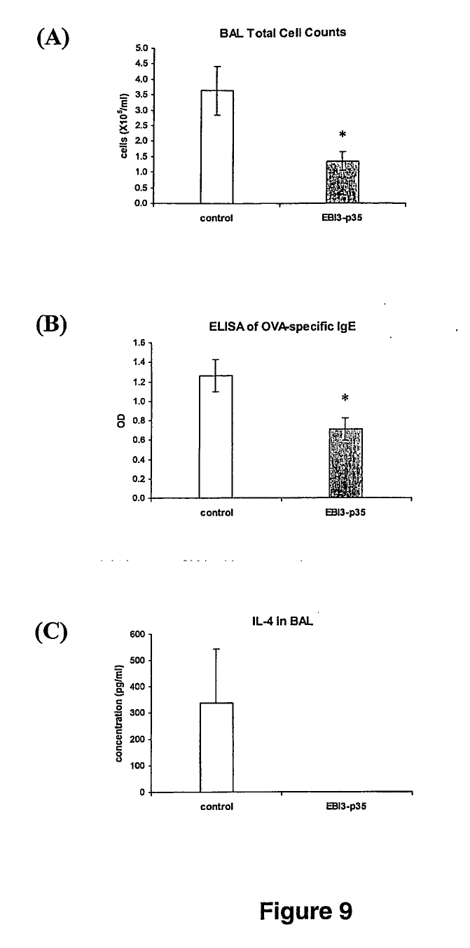

To confirm this, we tested EBI3-p35-Fc in a murine model of asthma. BALB/c mice were injected intraperitoneally with 100 μg of chicken ovalbumin (OVA) in an alum suspension on days 0 and 14. On day 14, mice were anaesthetised with avertin, and 100 μg of OVA in 40 μl of PBS was administered intratracheally (i.t.). Mice were again anaesthetised before being challenged i.t. on each of the days 25, 26, and 27 with 10 μg OVA in 40 μl of PBS. For EBI3-p35 treatment, mice were injected intraperitoneally with 2 μg of EBI3-p35-Fc on three occasions (day 25, 26 and 27) 1 h before the OVA challenge. Negative control mice were given PBS in place of OVA in both the sensitisation and the challenge stages. Mice were sacrificed on day 29 by administration of a fatal dose of avertin. Immediately after the administration of avertin, blood sample was collected by cardiac puncture. Bronchoalveolar lavage (BAL) was then harvested and counted using a haemocytometer. Total cell counts are shown in FIG. 9(A). Cytospin preparations were made and stained with Diff-Quick, in a rapid Romanowsky staining method to. Differential cell counting was performed using standard morphological criteria to determine the level of eosinophilia. The concentrations of ovalbumin-specific IgE and IL-4 in the BAL supernatant was determined by specific ELISA.

FIG. 9 shows that mice treated with EBI3-p35-Fc produced markedly reduced total BAL cellular infiltrates and importantly significantly reduced eosinophila (data not shown), serum OVA-specific IgE, and total IL-4 in the BAL fluid. All of these are hallmarks of airway hypersensitivity. These results demonstrate that EBI3-p35 is effective in attenuating an established asthma.

### 9. Production and Characterization of Human EBI3-p35

Human EBI3-p35 has now also been cloned as a fusion protein with Fc. Its activity in vitro has been tested using human peripheral blood T cells. The human EBI3-p35-Fc protein duplicates all the properties of the murine EBI3-p35-Fc described in Section 6 above and depicted in FIGS. 2 to 5 (data not shown).

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All references cited herein are expressly incorporated by reference.

1. A method of stimulating proliferation of a regulatory T cell, comprising contacting the cell with EBI3-p35.

2. A method according to claim 1 wherein the EBI3-p35 comprises at least two EBI3 components and two p35 components.

3. A method according to claim 2 wherein the EBI-p35 is a heterotetramer consisting of two of each component.

4. A method according to claim 2 wherein at least one EBI3 component and at least one p35 component are covalently linked to one another.

5. A method according to claim 4 wherein the at least one EBI3 component and the at least one p35 component form a fusion protein.

6. A method according to claim 4 wherein each EBI3 or p35 component is covalently linked to at least one other such component.

7. A method according to claim 1 wherein the EBI3-p35 further comprises one or more heterologous polypeptides covalently linked to one or more of the EBI3 or p35 components.

8. A method according to claim 7 wherein two or more said heterologous polypeptides associate with one another to assist in the association between the EBI3 and p35 components.

9. A method according to claim 8 wherein the heterologous polypeptides associate with one another via disulphide bonds.

10. A method according to claim 9 wherein the heterologous polypeptides are antibody Fc regions including hinge regions.

11. A method according to claim 1 further comprising contacting the regulatory T cell with a substance capable of stimulating signalling through the cell's T cell receptor.

12. A method of enhancing regulatory T cell activity in a subject, comprising administering a medicament containing EBI3-p35 to that subject.

13. (canceled)

14. (canceled)

15. The method as claimed in claim 12, wherein the medicament is for the treatment of a condition characterised by inappropriate or undesirable T cell activation.

16. The method as claimed in claim 15, wherein the condition is an inflammatory or autoimmune disease.

17. The method as claimed in claim 16, wherein the condition is arthritis (e.g. rheumatoid arthritis), gastritis, pernicious anaemia, thyroiditis, insulitis, diabetes, sialoadenitis, adrenalitis, orchitis/oophoritis, glomerulonephritis, experimental autoimmune encephalitis, multiple sclerosis, chronic obstructive pulmonary disease, atherosclerosis or inflammatory bowel disease.

18. The method as claimed in claim 15 wherein the medicament is for the prevention or amelioration of allograft rejection.

19. The method as claimed in claim 15 wherein the condition is an allergy.

20. The method as claimed in claim 19 wherein the condition is asthma.

21. An EBI3-p35 molecule comprising an EBI3 component, a p35 component, and a heterologous component, wherein two or more such heterologous components are capable of associating with one another such that two or more such EBI-p35 molecules form a complex.

22. A molecule according to claim 21 wherein the EBI3, p35 and heterologous components form a fusion protein.

23. A molecule according to claim 21 wherein the heterologous components are capable of associating with one another by formation of disulphide bonds.

24. A molecule according to 21 wherein the heterologous component is an antibody Fc domain including the hinge region.

25. EBI3-p35 as claimed in claim 21 comprising two EBI3 components and two p35 components.

26. EBI3-p35 according to claim 25 wherein each of the EBI3 and p35 components is covalently linked to at least one other such component.

27. EBI3-p35 according to claim 25 further comprising one or more heterologous components.

28. EBI3-p35 according to claim 27 wherein at least one of each of the EBI3, p35 and heterologous components form a fusion protein.

29. A nucleic acid encoding a fusion protein according to claim 22.

30. An expression vector comprising a nucleic acid according to claim 29.

31. A host cell comprising an expression vector according to claim 30.
